(19) United States
(12) Patent Application Publication
(10) Pub. No.: US 2025/0111949 A1
(43) Pub. Date: Apr. 3, 2025

(54) METHODS AND SYSTEMS FOR ASSIGNING ALZHEIMER’S RISK SCORE TO A CANDIDATE PATIENT USING ALZHEIMER’S IDENTIFICATION PLATFORM

(71) Applicant: HC1 Insight LLC, Indianapolis, IN (US)

(72) Inventors: Bradley A. Bostic, Indianapolis, IN (US); Charles J. Clarke, Indianapolis, IN (US); William E. Robinson, Indianapolis, IN (US); Charles Girard, JR., Indianapolis, IN (US)

(73) Assignee: HC1 Insight LLC, Indianapolis, IN (US)

(21) Appl. No.: 18/903,574

(22) Filed: Oct. 1, 2024

Related U.S. Application Data
(60) Provisional application No. 63/542,117, filed on Oct. 3, 2023.

(51) Int. Cl. G16H 50/30; A61B 5/00; G16H 10/40; G16H 50/20

(52) U.S. Cl. CPC G16H 50/30; A61B 5/4088; G16H 10/40; G16H 50/20

(57) ABSTRACT

A computer system and computer implemented method for determining a risk level of a candidate patient for developing Alzheimer's disease are provided. Laboratory test results from a laboratory are received at a computing device having one or more processors. The laboratory test results correspond to the candidate patient. Prescription date indicative of medications taken by the candidate patient are received at the computing device. Diagnosis data indicative of medical diagnoses associated with the candidate patient are received at the computing device. Age and gender associated with the candidate patient are received at the computing device. Features of the laboratory test results are preprocessed into categories. An Alzheimer's risk score associated with the candidate patient is generated by the computing device utilizing at least one machine learning model based on the prescription data, diagnosis data, age, gender and categorized features. The Alzheimer's risk score is output by the computing device.

### CROSS-REFERENCE TO RELATED APPLICATIONS

This application claims priority to U.S. provisional application number 63/542,117 filed on Oct. 3, 2023. The above application is hereby incorporated by reference as if fully set forth herein in its entirety.

### FIELD

The present disclosure relates to an Alzheimer's identification platform that assigns an Alzheimer's risk score to a candidate patient using machine learning techniques that identifies risk factors that correlate with an Alzheimer's disease diagnosis.

BACKGROUND

Alzheimer's disease is a progressive disease that starts with mild memory loss and can lead to loss of the ability to carry on a conversation and/or respond to environmental inputs. With the first true treatments for Alzheimer's disease on the horizon, increased emphasis is being placed on how to most effectively identify patients who would benefit from future treatments. Treatments can include medications that tend to reverse the buildup of amyloid plaque in the brain and slow cognitive decline. Such medications are best administered early in the course of an Alzheimer's disease diagnosis. Current diagnostic protocols however rely on specific and expensive confirmatory tests performed under the care of a neurologist. As a result, patients often do not undergo screening until they begin to present symptoms of cognitive decline.

A need exists for improved methods and systems for a pre-screening tool that can identify pre-symptomatic individuals who are at elevated risk for developing Alzheimer's disease.

### SUMMARY

Improved methods, systems, components, processes, modules, and other elements (collectively referred to alternatively herein as the “Alzheimer's identification platform,” or simply as the “platform”) for assigning an Alzheimer's risk score to an individual is provided.

According to some embodiments of the present disclosure, a computer system and computer implemented method for determining a risk level of a candidate patient for developing Alzheimer's disease are provided. Laboratory test results from a laboratory are received at a computing device having one or more processors. The laboratory test results correspond to the candidate patient. Prescription data indicative of medications taken by the candidate patient are received at the computing device. Diagnosis data indicative of medical diagnoses associated with the candidate patient are received at the computing device. Age and gender associated with the candidate patient are received at the computing device. Features of the laboratory test results are preprocessed into categories. An Alzheimer's risk score associated with the candidate patient is generated by the computing device utilizing at least one machine learning model based on the prescription data, diagnosis data, age, gender and categorized features. The Alzheimer's risk score is output by the computing device.

In examples, the laboratory test results are indicative of at least one blood test of the candidate patient. The prescription data can be sourced from a pharmacy system, an electronic medical records system, or from claims data. The diagnosis data can be sourced from at least one of a healthcare system and an insurance system. The preprocessing generates at least one of an average, median, minimum and maximum value of the categorized features. The categorized features can relate to at least one of alanine transaminase, estimated glomerular filtration rate, hemoglobin, and hematocrit.

In additional examples, the diagnosis data includes medical diagnosis related to at least one of anemia, aphasia, atherosclerosis, cerebrovascular disease, chest pain, chronic kidney disease, diseases of the heart, mobility, hearing loss, hypertension, and hypokalemia.

In other examples, at least one machine learning model includes a first model representative of females aged 50-64, a second model representative of females aged 65-80, a third model representative of females aged over 80, a fourth model representative of males aged 50-64, a fifth model representative of males aged 65-80; and a sixth model representative of males aged over 80.

A more complete understanding of the disclosure will be appreciated from the description and accompanying drawings and the claims, which follow.

### BRIEF DESCRIPTION OF THE DRAWINGS

The accompanying drawings, which are included to provide a better understanding of the disclosure, illustrate embodiment(s) of the disclosure and, together with the description, serve to explain the principle of the disclosure. In the drawings:

FIG. 1 is a schematic illustrating an example environment of an Alzheimer's identification platform according to some embodiments of the present disclosure;

FIG. 2 is a flow diagram illustrating steps for generating models according to some embodiments of the present disclosure;

FIG. 3 is flow diagram illustrating an example method of using a machine learning system that implements the models of FIG. 2 to output an Alzheimer's score associated with a candidate patient;

FIG. 4 is a diagram of an example computing system including an example computing device and an example server computing device according to some implementations of the present disclosure; and

FIG. 5 is a functional block diagram of the example computing device of FIG. 4.

### DETAILED DESCRIPTION

As mentioned above, there is a need for improved methods and systems for identifying individuals who are at elevated risk for developing Alzheimer's disease (AD). In order to address current challenges in the identification of AD, including the limited capacity of neurologists to screen patients, the expense of current diagnostics, and the lack of efficient cost-effective tools for health systems to pre-screen patients for early signals of risk for developing AD, the present disclosure is directed to an AD identification platform that identifies individuals who are at elevated risk for developing AD. The AD identification platform can be utilized as a pre-screening tool to perform various functions, including but not limited to outputting an Alzheimer's risk score that can be used to identify individuals within a population who are at elevated risk for developing AD. These patients may be prioritized for further screening, either in a primary care setting or under the care of a neurologist.

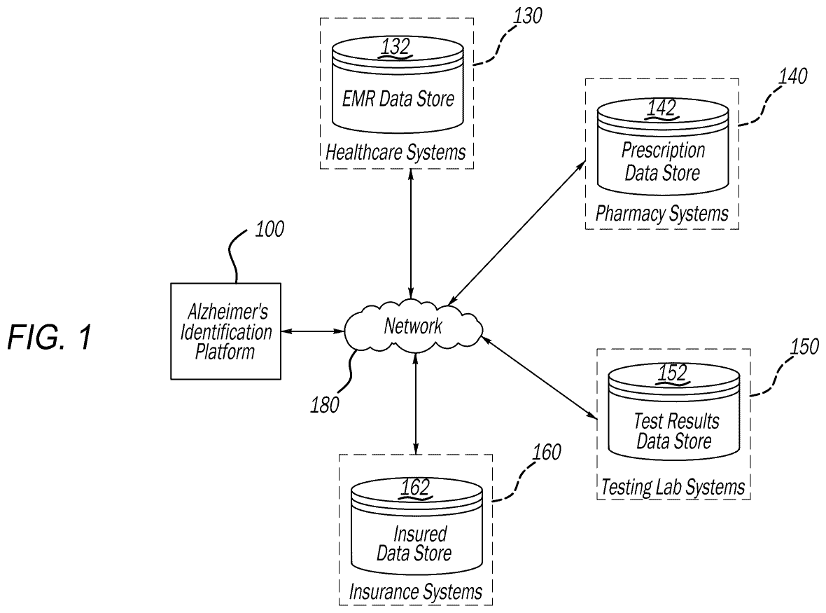

FIG. 1 illustrates an example Alzheimer's identification platform 100 according to some embodiments of the present disclosure. In embodiments, the Alzheimer's identification platform 100 is configured to collect and monitor data from multiple external sources, including electronic medical records (EMRs) from healthcare systems 130, prescription drug records from pharmacy systems 140, test results from testing lab systems 150 and insurance data from insurance systems 160. In examples, the healthcare systems 130 can provide EMR sourced from an EMR data store 132, the pharmacy systems 140 can provide prescription drug records sourced from a prescription data store 142, the testing lab systems 150 can provide test data results from a test results data store 152, and the insurance systems 160 can provide insurance data sourced from an insured data store 162. It will be appreciated that data can additionally or alternatively be collected from other external sources.

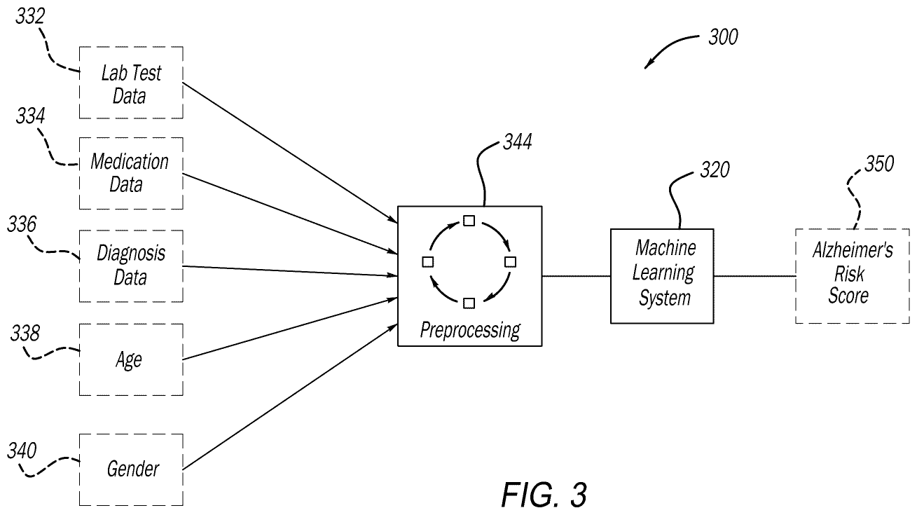

In embodiments, the Alzheimer's identification platform 100 may include a machine learning system 320 (FIG. 3) that is configured to train machine learned models that are leveraged by the Alzheimer's identification platform 100. The machine learning system 320 may train any suitable type of model, including neural networks, deep neural networks, recurrent neural networks, Hidden Markov Models, Bayesian models, regression models, and the like. The models can be estimated using algorithms such as, but not limited to, logistic regression, extreme gradient boosting, support vector machines, naïve bayes nearest neighbor, decision trees, and random forest. The machine learning system 320 may train the models in a supervised, unsupervised, or semi-supervised manner. In embodiments, the machine learning system 320 may collect training data from one or more data sources such as the heathcare systems 130, the pharmacy systems 140, the testing lab systems 150 and the insurance systems 160. The training data can include an oversampled cohort of patients with a confirmed AD diagnosis. In this regard, the training data can be based on objective clinical information that is commonly captured in a primary care setting.

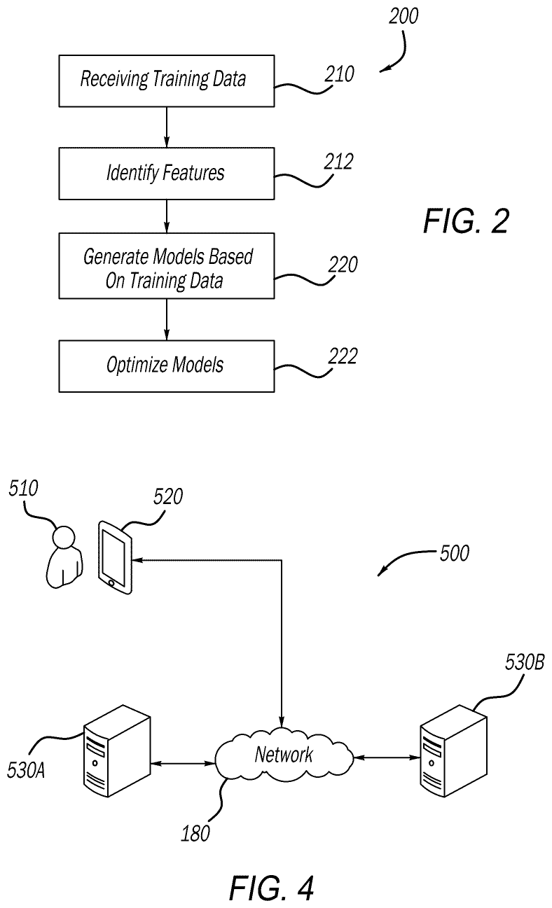

With additional reference now to FIG. 2, a flow diagram 200 illustrating an example method of generating models used in the machine learning system 320 according to some embodiments of the present disclosure is shown. At 210 training data is received. The training data includes some of the data from at least one of the EMR data store 132, the prescription data store 142, the test results data store 152 and the insured data store 162 that can be compiled as lab test data, medication data, other diagnosis data, age data and gender data. At 212 features are identified from the data. By way of example, various features identified are shown in Table 1 below. The features can be categorized generally by lab test data, medication data, and other diagnosis data. Age is also identified as a feature.

TABLE 1

| FEATURE | CATEGORY |
|---|---|
| AGE | Age |
| Anemia | Other Diagnosis |
| Aphasia /Slurred Speech | Other Diagnosis |
| Atherosclerosis | Other Diagnosis |
| AVG_ALT | Lab Test Data |
| AVG_EGFR_AA | Lab Test Data |
| AVG_EGFR_NON_AA | Lab Test Data |
| AVG_HEMATOCRIT | Lab Test Data |
| AVG_HEMOGLOBIN | Lab Test Data |
| Carbidopa and Levodopa | Medications |
| Cerebrovascular disease | Other Diagnosis |
| Chest Pain | Other Diagnosis |
| Chronic Kidney Disease | Other Diagnosis |
| Coordination | Other Diagnosis |
| Dehydration | Other Diagnosis |
| Depressive | Other Diagnosis |
| Diabetes | Other Diagnosis |
| Diseases of arteries; arterioles; | Other Diagnosis |
| and capillaries | |
| Diseases of the heart | Other Diagnosis |
| Diseases of the urinary system | Other Diagnosis |
| Divalproex Sodium | medications |
| Falling | Other Diagnosis |
| Gait/Mobility | Other Diagnosis |
| Head/Brain Injury | Other Diagnosis |
| Hearing Loss | Other Diagnosis |
| Heart Failure | Other Diagnosis |
| Hyperlipidemia | Other Diagnosis |
| Hypertension | Other Diagnosis |
| Hypokalemia | Other Diagnosis |
| Hypo-osmolality / | Other Diagnosis |
| Hyponatremia | |
| Hypotension | Other Diagnosis |
| MAX_ALT | Lab Test Data |
| MAX_EGFR_AA | Lab Test Data |
| MAX_EGFR_NON_AA | Lab Test Data |
| MAX_HEMOGLOBIN | Lab Test Data |
| MEDIAN_ALT | Lab Test Data |
| MEDIAN_EGFR_AA | Lab Test Data |
| MEDIAN_EGFR_NON_AA | Lab Test Data |
| MEDIAN_HEMATOCRIT | Lab Test Data |
| MEDIAN_HEMOGLOBIN | Lab Test Data |
| MIN_EGFR_AA | Lab Test Data |
| MIN_EGFR_NON_AA | Lab Test Data |
| MIN_HEMATOCRIT | Lab Test Data |
| MIN_HEMOGLOBIN | Lab Test Data |
| Parkinson | Other Diagnosis |
| Quetiapine | Medications |
| Quetiapine Fumarate | Medications |
| Restlessness | Other Diagnosis |
| Tremor | Other Diagnosis |
| Urinary tract infection | Other Diagnosis |
| Vascular Disease | Other Diagnosis |
| White Matter | Other Diagnosis |

As identified in Table 1, “ALT” is used to denote “alanine transaminase”; “EGFR” is used to denote “estimated glomerular filtration rate”; “AA” is used to denote “African American”; 1 AVG” is used to denote “average”; “Max” is used to denote “maximum”; “MIN” is used to denote “minimum”.

At 220 models are generated based on the training data. In examples, the models were developed specifically for application to early stages of AD. In this regard, patient data that was collected after AD diagnosis were excluded as input factors to training the models. In examples, the models assign an importance score to each of the features. The features or risk factors that correlate with an AD diagnosis have elevated importance scores. In examples, factors that were closely associated with AD or dementia diagnosis and treatment were broadly excluded from the models since these indicate that a patient is currently under the care of a physician for those diseases and is therefore outside of the “pre-diagnosis” window. In this regard, the models are generally based on correlations that exist prior to the diagnosis of the AD.

As described herein, various models can be generated based on age and gender. In examples, various models are generated and categorized by gender and age. For example, various models generated include: Females aged 50-64, Females aged 65-80, Females aged over 80, Males aged 50-64, Males aged 65-80 and Males aged over 80. It will be appreciated that additional models may be generated that are categorized differently. At 222, the models are optimized. In examples, the models are tested against additional data from the data stores of patients with confirmed AD diagnosis and not used in the training data. In examples, the models were also validated using a normal, non-oversampled dataset, in which AD is relatively rare. In additional examples, the models can be refined to determine an effective diagnosis date based upon various factors (diagnosis codes, start date of a prescription drug therapy, first consultation with a neurologist, etc.). The models can be further expanded to include other key factors provided in electronic heath records such as related to race, ethnicity, and family history. The models can be further tuned based on the use case to strike an optimal balance between sensitivity and specificity.

By way of example, the dataset used to generate the models is shown in Table 2 below. It is appreciated however that other data can be used to generate the models within the scope of the present disclosure.

TABLE 2
Overall Data

| | |
|---|---|
| 58,000 Alzheimer's patients diagnosed in | 143,000 patients with no AD or Dementia |
| the past 2 years over the age of 50 | diagnosis |
| 62% Female, average age of 79 | 51% Female, average age of 70 |

| | | | |
|---|---|---|---|
| Men 65-80 | Women 65-80 | Men 80+ | Women 80+ |

Training Data

| | | | |
|---|---|---|---|
| 35,306 | 40,046 | 17,844 | 26,070 |
| 22.9% AD | 30% AD | 47.8% AD | 42.4% AD |

Validation Data

| | | | |
|---|---|---|---|
| 7,168 | 7,678 | 2,522 | 3,251 |
| 4.9% AD | 9.1% AD | 7.1% AD | 14% AD |

With additional reference now to FIG. 3, a flow diagram 300 illustrating an example method of using the machine learning system 320 implemented by the Alzheimer's identification platform 100 is shown. The machine learning system 320 used to generate the models described in FIG. 2 is used to output an Alzheimer's risk score 350 for a candidate patient. Data inputs collectively identified at 330, for the candidate patient are input into a preprocessing module 344. The data inputs 330 can include lab test data 332, medication data 334, diagnosis data 336, age data 338 and gender data 340 associated with the candidate patient. In examples, the preprocessing module 344 can process raw data into usable features consistent for use with the machine learning system 320. For example, various lab test data can be preprocessed to generate desired “average”, “median”, “minimum” and “maximum” values shown in the example features listed in Table 1.

The preprocessing module 344 sends processed data to the machine learning system 320. In examples, the data inputs 330 can include lab test data 332, medication data 334, diagnosis data 336, age data 338 and gender data 340 specific to the candidate patient. The machine learning system 320 compares the processed data output from the preprocessing module 344 related to the candidate patient to the generated models and determines the Alzheimer's risk score 350 specific to the candidate patient. By way of example only, the model used by the machine learning system is the model that satisfies the age and gender of the candidate patient. Explained further, if the candidate patient is a 67 year old woman, the data of the candidate patient is used in the model representing females aged 65-80. The Alzheimer's identification platform 100 may perform additional or alternative tasks without departing from the scope of the disclosure.

Returning to FIG. 1, the Alzheimer's identification platform 100 may communicate with external sources such as electronic medical records (EMRs), insurer databases, pharmacy databases, testing lab databases, and/or prescription drug monitoring programs, as well as other computing device(s), systems, data sources, applications, and platforms, via a network 180. It should be appreciated that the network 180 can take the form of any communication network suitable for communicatively linking computing devices and/or components thereof, including, without limitation, a virtual private network, the Internet, a Local Area Network, a Wide Area Network, a cellular network, and an intranet or other private network.

In embodiments, the Alzheimer's identification platform 100 may provide notifications and/or recommendations to appropriate third parties, such as healthcare organizations (e.g., hospitals and/or clinics), physicians, pharmacies, insurers, and the like. In some of these embodiments, the platform 100 may provide customer relationship management capabilities, whereby the platform 100 may leverage these capabilities to provide the notifications and/or recommendations.

A healthcare system 130 may refer to a collection of one or more computing devices, including client user devices and/or server devices that are used in connection with a healthcare organization (e.g., one or more hospitals, doctor offices, etc.). In embodiments, a healthcare system 130 may include an EMR data store 132. An EMR data store 132 may include one or more databases that store and/or index electronic medical records. A respective electronic medical record may store or reference patient data of a respective patient of the healthcare organization. An electronic medical record may include a patient identifier, one or more physician identifiers that indicate the respective physicians of a patient, physician notes relating to the patient, prescription data indicating treatments that were prescribed to a patient, test results of the patient, and the like. The EMR data store 132 may store additional or alternative data without departing from the scope of the disclosure.

A pharmacy system 140 may refer to a collection of one or more computing devices, including client user devices and/or server devices that are used in connection with a pharmacy organization (e.g., a pharmacy or chain of pharmacies). In embodiments, the pharmacy system 140 may include a prescription data store 142. The prescription data store 142 may include one or more databases that store and/or index prescription records. A respective prescription record may store a prescription ID that uniquely identifies a prescription of a patient, a patient ID identifying the patient to whom the prescription corresponds, a physician ID that identifies the physician that wrote the prescription, a medication ID that identifies the medication that was prescribed, a quantity of the medication that is prescribed, a dosage of the medication that is prescribed, a date on which the medication was prescribed, a date on which the prescription expires, and the like. The prescription data store 142 may store additional or alternative data without departing from the scope of the disclosure.

A testing lab system 150 may refer to a collection of one or more computing devices, including client user devices and/or server devices that are used in connection with an organization that performs medical testing (e.g., blood testing, a urine analysis, genetic testing, and the like). In embodiments, the testing lab system 150 may include a test results data store 152. The test results data store 152 may include one or more databases that store and/or index test result records that respectively correspond to testing administered by the organization. In embodiments, a test results record may include a test ID that identifies the test that was performed, a test type ID that identifies the type of test performed, a subject ID that indicates a subject of the test (e.g., a patient), a requestor ID that indicates an organization that ordered the test (e.g., a healthcare organization or physician), results data (e.g., the results of the test), and a date (e.g., a date on which the test was administered). The test results data store 152 may store additional or alternative data without departing from the scope of the disclosure.

An insurance system 160 may refer to a collection of one or more computing devices, including client user devices and/or server devices that are used in connection with an insurance organization (e.g., a health insurance provider). The insurance system 160 may be configured to process claims, payout medical bills, collect medical data relating to insured patients, and the like. In embodiments, an insurance system 160 may include an insured data store 162. The insured data store 162 may include one or more databases that store and/or index insured records. A respective insured record corresponds to a respective insured person and may store an insured ID that uniquely identifies the person being insured, a policy ID that identifies the policy of the insured, one or more healthcare system IDs that identify one or more respective healthcare systems that the insured visits or has visited, one or more physician IDs that respectively identify a physician of the insured, one or more prescription IDs that respectively identify one or more respective prescriptions of the insured, billing information of the patient, a medical history of the patient, and the like. The insured data store 1622 may store additional or alternative data without departing from the scope of the disclosure.

In embodiments, the Alzheimer's identification platform 100 may utilize machine learning and AI techniques to determine if a candidate patient is at an elevated risk for developing AD. For example, in embodiments, various machine learned models may be trained to identify when a patient is at an elevated risk for developing AD. These models may be trained on training data samples relating to patients that were determined to have an AD diagnosis. In these embodiments, the Alzheimer's identification platform 100 may obtain lab test results of the patient (e.g., a blood test and/or a urine analysis test), a prescription medication that the patient is being prescribed, and relevant patient information (e.g., an ailment of the patient, other prescriptions of the patient, an age of the patient, a gender of the patient, a weight of the patient, a body fat percentage of the patient, and the like). In embodiments, the machine learned models can be categorized by way of gender and age. Other machine learned models categorized in other ways may be provided. The gender and age of the candidate patient is used to determine which machine learned model to use for generating the Alzheimer's risk score 350. The Alzheimer's risk score 350 can be used to indicate whether the candidate patient is at an elevated risk for developing AD.

Referring now to FIG. 4, a diagram of an example computing system 500 is illustrated. The computing system 500 can be configured to implement the Alzheimer's identification platform 100 described herein, e.g., amongst a plurality of users 510 via their computing devices 520. The computing system 500 can include one or more example computing devices 520 and one or more example servers 530 that communicate via a network 180 (as described above) according to some implementations of the present disclosure. For ease of description, in this application and as shown in FIG. 4, one example computing device 520 and two example server computing devices 530 (server computing devices 530A and 530B) are illustrated and described. It should be appreciated, however, that there can be more computing devices 500 and more or less server computing devices 530 than is illustrated. While illustrated as a mobile phone (a “smart” phone), each computing device 520 can be any type of suitable computing device, such as a desktop computer, a tablet computer, a laptop computer, a wearable computing device such as eyewear, a watch or other piece of jewelry, or clothing that incorporates a computing device.

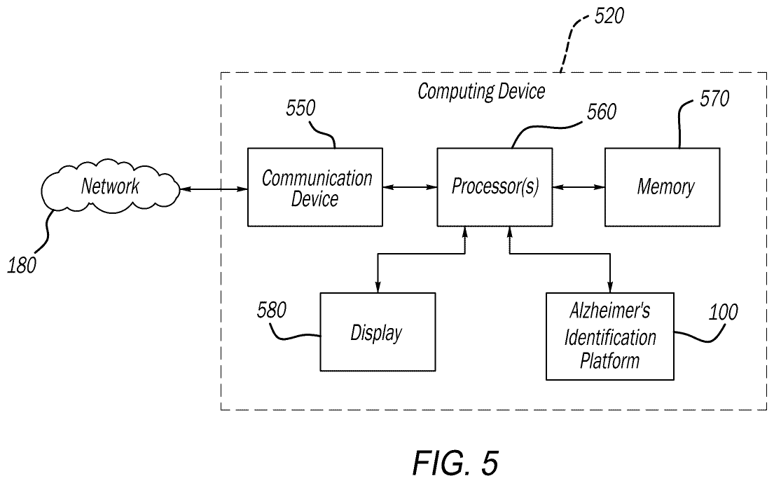

A functional block diagram of an example computing device 520 is illustrated in FIG. 5. The computing device 520 is shown as including a communication device 550, one more processors 560, a memory 570, a display device 580, and the Alzheimer's identification platform 100. The processor(s) 560 can control the operation of the computing device 520, including implementing at least a portion of the techniques of the present disclosure. The term “processor” as used herein is intended to refer to both a single processor and multiple processors operating together, e.g., in a parallel or distributed architecture.

The communication device 550 can be configured for communication with other devices (e.g., the server computing devices 530 or other computing devices 520) via the network 180. One non-limiting example of the communication device 550 is a transceiver, although other forms of hardware are within the scope of the present disclosure. The memory 570 can be any suitable storage medium (flash, hard disk, etc.) configured to store information. For example, the memory 570 may store a set of instructions that are executable by the processor 560, which causes the computing device 520 to perform operations (e.g., such as the operations of the present disclosure). The display device 580 can display information to the user 510. In some implementations, the display device 580 can comprise a touch-sensitive display device (such as a capacitive touchscreen and the like), although non-touch display devices are within the scope of the present disclosure.

It should be appreciated that the example server computing devices 530 can include the same or similar components as the computing device 520, and thus can be configured to perform some or all of the techniques of the present disclosure. Further, while the techniques of the present disclosure are described herein in the context of the Alzheimer's identification platform 100, which is illustrated as being a component of the computing device 520, it is specifically contemplated that each feature of the techniques may be performed by a single computing device 520 alone, a plurality of computing devices 520 operating together, a server computing device 530 alone, a plurality of server computing devices 530 operating together, and a combination of one or more computing devices 520 and one or more server computing devices 530 operating together.

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The terms “a” or “an,” as used herein, are defined as one or more than one. The term “another,” as used herein, is defined as at least a second or more. The terms “including” and/or “having,” as used herein, are defined as comprising (i.e., open transition).

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entirety to the full extent permitted by law.

The methods and systems described herein may be deployed in part or in whole through a machine that executes computer software, program codes, and/or instructions on a processor. The present disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platforms. A processor may be any kind of computational or processing device capable of executing program instructions, codes, binary instructions and the like. The processor may be or may include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor, or any machine utilizing one, may include non-transitory memory that stores methods, codes, instructions and programs as described herein and elsewhere. The processor may access a non-transitory storage medium through an interface that may store methods, codes, and instructions as described herein and elsewhere. The storage medium associated with the processor for storing methods, programs, codes, program instructions or other types of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, Internet server, intranet server, cloud server, and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server. In embodiments, the server may be a virtual machine that is executed by a processing system of a cloud-services platform (e.g., Amazon AWS). In these embodiments, the cloud-services platform may offer computing resources that host and support various aspects of a third-party's software systems.

The server may provide an interface to other devices including, without limitation, clients, other servers, printers, database servers, print servers, file servers, communication servers, distributed servers, social networks, and the like. Additionally, this coupling and/or connection may facilitate remote execution of programs across the network. The networking of some or all of these devices may facilitate parallel processing of a program or method at one or more location without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, Internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of programs across the network. The networking of some or all of these devices may facilitate parallel processing of a program or method at one or more location without deviating from the scope of the disclosure. In addition, any of the devices attached to the client through an interface may include at least one storage medium capable of storing methods, programs, applications, code, and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, and instructions described herein and elsewhere may be executed by one or more of the network infrastructural elements. The methods and systems described herein may be adapted for use with any kind of private, community, or hybrid cloud computing network or cloud computing environment, including those that involve features of software as a service (SaaS), platform as a service (PaaS), and/or infrastructure as a service (IaaS).

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic book readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or intangible items from one state to another. The methods and systems described herein may also transform data representing physical and/or intangible items from one state to another.

The elements described and depicted herein, including in flowcharts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flowchart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable devices, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium. The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, methods described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples but is to be understood in the broadest sense allowable by law.

The use of the terms “a” and “an” and “the” and similar referents in the context of describing the disclosure (especially in the context of the following claims) is to be construed to cover both the singular and the plural unless otherwise indicated herein or clearly contradicted by context. The terms “comprising,” “having,” “including,” and “containing” are to be construed as open-ended terms (i.e., meaning “including, but not limited to,”) unless otherwise noted. Recitations of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the foregoing written description enables one skilled in the art to make and use what is considered presently to be the best mode thereof, those skilled in the art will understand and appreciate the existence of variations, combinations, and equivalents of the specific embodiment, method, and examples herein. The disclosure should therefore not be limited by the above-described embodiment, method, and examples, but by all embodiments and methods within the scope and spirit of the disclosure.

Any element in a claim that does not explicitly state “means for” performing a specified function, or “step for” performing a specified function, is not to be interpreted as a “means” or “step” clause as specified in 35 U.S.C. § 112(f). In particular, any use of “step of” in the claims is not intended to invoke the provision of 35 U.S.C. § 112(f).

Persons skilled in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A computer implemented method for determining a risk level of a candidate patient for developing Alzheimer's disease, comprising:
receiving, at a computing device having one or more processors, laboratory test results from a laboratory, the laboratory test results corresponding to the candidate patient;
receiving, at the computing device, prescription data indicative of medications taken by the candidate patient;
receiving, at the computing device, diagnosis data indicative of medical diagnoses associated with the candidate patient;
receiving, at the computing device, an age and gender associated with the candidate patient;
preprocessing, by the computing device, the laboratory test results thereby categorizing features of the laboratory test results;
generating, by the computing device, an Alzheimer's risk score associated with the candidate patient utilizing at least one machine learning model based on the prescription data, diagnosis data, age, gender and categorized features; and
outputting, by the computing device, the Alzheimer's risk score.

2. The computer implemented method of claim 1 wherein the laboratory test results are indicative of at least one blood test of the candidate patient.

3. The computer implemented method of claim 1 wherein the prescription data is sourced from a pharmacy system.

4. The computer implemented method of claim 1 wherein the diagnosis data is sourced from at least one of a healthcare system and an insurance system.

5. The computer implemented method of claim 1 wherein the preprocessing generates at least one of an average, median, minimum and maximum value of the categorized features.

6. The computer implemented method of claim 1 wherein the categorized features relate to at least one of alanine transaminase, estimated glomerular filtration rate, hemoglobin, and hematocrit.

7. The computer implemented method of claim 1 wherein the diagnosis data includes medical diagnosis related to at least one of anemia, aphasia, atherosclerosis, cerebrovascular disease, chest paid, chronic kidney disease, diseases of the heart, mobility, hearing loss, hypertension, and hypokalemia.

8. The computer implemented method of claim 1 wherein the at least one machine learning model includes a first model representative of females aged 50-64, a second model representative of females aged 65-80, a third model representative of females aged over 80, a fourth model representative of males aged 50-64, a fifth model representative of males aged 65-80; and a sixth model representative of males aged over 80.

9. A computing system, comprising:
one or more processors; and
a non-transitory computer-readable storage medium having a plurality of instructions stored thereon, which, when executed by the one or more processors, cause the one or more processors to perform operations comprising:
receiving, at a computing device having one or more processors, laboratory test results from a laboratory, the laboratory test results corresponding to the candidate patient;
receiving, at the computing device, prescription data indicative of medications taken by the candidate patient;
receiving, at the computing device, diagnosis data indicative of medical diagnoses associated with the candidate patient;
receiving, at the computing device, an age and gender associated with the candidate patient;
preprocessing, by the computing device, the laboratory test results thereby categorizing features of the laboratory test results;
generating, by the computing device, an Alzheimer's risk score associated with the candidate patient utilizing at least one machine learning model based on the prescription data, diagnosis data, age, gender and categorized features; and
outputting, by the computing device, the Alzheimer's risk score.

10. The computing system of claim 9 wherein the laboratory test results are indicative of at least one blood test of the candidate patient.

11. The computing system of claim 9 wherein the prescription data is sourced from a pharmacy system.

12. The computing system of claim 9 wherein the diagnosis data is sourced from at least one of a heathcare system and an insurance system.

13. The computing system of claim 9 wherein the preprocessing generates at least one of an average, median, minimum and maximum value of the categorized features.

14. The computing system of claim 9 wherein the categorized features relate to at least one of alanine transaminase, estimated glomerular filtration rate, hemoglobin, and hematocrit.

15. The computing system of claim 9 wherein the diagnosis data includes medical diagnosis related to at least one of anemia, aphasia, atherosclerosis, cerebrovascular disease, chest paid, chronic kidney disease, diseases of the heart, mobility, hearing loss, hypertension, and hypokalemia.

16. The computing system of claim 9 wherein the at least one machine learning model includes a first model representative of females aged 50-64, a second model representative of females aged 65-80, a third model representative of females aged over 80, a fourth model representative of males aged 50-64, a fifth model representative of males aged 65-80; and a sixth model representative of males aged over 80.
